(19) United States
(12) Patent Application Publication
(10) Pub. No.: US 2021/0138461 A1
(43) Pub. Date: May 13, 2021

(54) BIOSENSOR CARTRIDGE WITH SAMPLE ACQUISITION

(71) Applicant: QORVO US, INC., Greensboro, NC (US)

(72) Inventors: Thayne L. Edwards, Bend, OR (US); Florian Bell, Bend, OR (US)

(21) Appl. No.: 16/319,540

(22) PCT Filed: Aug. 2, 2017
(86) PCT No.: PCT/US2017/045184

§ 371 Date: Jan. 22, 2019

Related U.S. Application Data
(60) Provisional application No. 62/369,882, filed on Aug. 2, 2016.

(51) Int. Cl. B01L 3/00

(52) U.S. Cl. CPC B01L 3/502715; B01L 2200/027; B01L 2200/028; B01L 2200/0684; B01L 2400/0487; B01L 2300/0672; B01L 2300/165; B01L 2300/0819; B01L 2400/0406; B01L 2300/0663

(57) ABSTRACT

A sampler for acquiring and transporting sample material may include a sampler body defining a sampling volume configured to hold the sample material. The sampler body may define a sample port that may be used as an entrance to collect the sample material and an exit to expel the sample material. The sample port may be configured to be operably connected to a fluidic channel of a cartridge. The sampler may also define a pump connection port configured to be operably connected to a pump. The pump may force the sample material out of the sampling volume through the sample port.

### RELATED APPLICATIONS

This application claims priority to U.S. Provisional Application Ser. No. 62/369,882, filed on 2 Aug. 2016, the contents of which are incorporated herein in its entirety.

### TECHNICAL FIELD

Embodiments of the present disclosure relate generally to bulk acoustic wave (BAW) resonators and their use as biosensors. In particular, the present disclosure relates to cartridges containing bulk acoustic wave resonators and samplers that may be configurable therewith.

### BACKGROUND

Numerous instruments and measurement techniques exist for diagnostic testing of materials for medical, veterinary medical, environmental, biohazard, bioterrorism, agricultural, and food safety purposes. Diagnostic testing traditionally requires long response times to obtain meaningful data, involves expensive, remote, or cumbersome laboratory equipment, requires large sample size, utilizes multiple reagents, demands highly trained users, and can involve significant direct and indirect costs. For example, in both the human and veterinary diagnostic markets, most tests require that a sample be collected from a patient and then be sent to a laboratory, where the results are not available for several hours or days. As a result, the caregiver must wait to treat the patient.

Point of use (or point of care when discussing human or veterinary medicine) solutions for diagnostic testing and analysis, although capable of solving most of the noted drawbacks, remain somewhat limited. Even some of the point of use solutions that are available, are limited in sensitivity and reproducibility compared to in-laboratory testing. There are also often significant costs involved as separate systems may be needed for different point of use tests.

In addition, several steps may be involved from sample acquisition to testing. For example, the sample may be acquired, stored, transported to the testing site, manipulated, and transferred to the testing instrument.

It would be desirable to provide a biosensor platform for point of use testing that is simple and cost-effective to manufacture, allows for flexibility to test for various analytes on the same platform, and is convenient and reliable. It would also be desirable to provide a simple sample acquisition process that may be readily integrated with the testing instrument, or a portion thereof, to limit sample manipulation and transfer steps.

### SUMMARY

Embodiments described herein may provide integrated sample acquisition with a testing cartridge. Preferably, the testing cartridge includes a sensor comprising a bulk acoustic wave (BAW) resonator. BAW sensors tend to be very sensitive and employ a small volume of sample, and thus may be useful as point of use testing.

An illustrative sampler may include a sampler body extending between a distal end and a proximal end. The sampler body may include an inner surface defining a sampling volume within the sampler body that may be configured to hold a sample material. The sampler body may define a sample port proximate the proximal end and a pump connection port proximate the distal end. Each of the sample port and the pump connection port may be in communication with the sampling volume. The sample port may be configured to be operably connected to a fluidic channel of a cartridge. The pump connection port may be configured to be operably connected to a pump.

In one or more embodiments, the inner surface of the sampler body may include a hydrophilic material configured to draw the sample material into the sampling volume through the sample port. In one or more embodiments, the pump connection port may include a vent including a hydrophobic material such that the sample material is prevented from passing through the vent. In one or more embodiments, the sampler may be configured to force the sample material from the sampling volume through the sample port in response to pneumatic forces applied through the pump connection port by the pump. In one or more embodiments, the pneumatic forces applied to the pump connection port by the pump may force the sample material from the sampling volume at a controlled flow rate. In one or more embodiments, a total volume of the sampling volume may limit a total volume of the sample material held by the sampling volume. The total volume may be between about 10 microliters to 100 microliters. In one or more embodiments, the sampler may further include a puncture portion located proximate the sample port and configured to puncture a barrier.

An illustrative system may include the sampler as described above and a cartridge. The cartridge may include a cartridge body, a sensor, and a fluidic channel. The cartridge body may define a receptacle extending inwards from an outer surface of the cartridge body to a base end of the receptacle. The receptacle may be configured to receive the sampler such that the sampler may be removably couplable to the cartridge. The sensor may include a bulk acoustic wave resonator having a sensing surface. The fluidic channel may be configured to carry fluid at least between the receptacle and the sensor.

The fluidic channel may include a first fluidic channel portion, a second fluidic channel portion, a third fluidic channel portion, and a fourth fluidic channel portion. The first fluidic channel portion may extend between a reservoir connection port and the receptacle. The second fluidic channel portion may extend from the first fluidic channel portion and proximate the base end of the receptacle. The second fluidic channel portion may be in fluid communication with the sample port of the sampler when the sampler is received by the receptacle. The third fluidic channel portion may extend between the second fluidic channel portion and the sensor. The fourth fluidic channel portion may extend from the third fluidic channel portion and across the sensing surface of the resonator.

In one or more embodiments, at least a portion of the second fluidic channel portion may be formed by the sampler when the sampler is received by the receptacle. In one or more embodiments, the cartridge may include a fluid reservoir configured to hold a buffer material. The first fluidic channel portion may be in fluid communication with the fluid reservoir. The fluidic channel may be configured to carry the buffer material from the fluid reservoir to at least the sensor. The cartridge may further include the reservoir connection port in fluid communication with the fluid reservoir. In one or more embodiments, the pump may be operably coupled to the pump connection port to force the sample material out of the sampling volume and may be operably coupled to the reservoir connection port to force the buffer material through the fluidic channel. The system may also include a valve operably coupled between the pump and each of the pump connection port and the reservoir connection port. The valve may be configured to selectively control pressure from the pump to each of the sampling volume and the fluid reservoir. In one or more embodiments, the pump may be configured to apply a force through the pump connection port of the sampler to force the sample material out of the sampling volume at a controlled flow rate. In one or more embodiments, the sampler and the receptacle may form a seal when the sampler is received by the receptacle to form a fluid-tight interface between the sample port and the second fluidic channel portion.

An illustrative method of testing a sample material may include obtaining the sample material in a sampler, inserting a sampler body of the sampler into a receptacle of a cartridge, and forcing the sample material out of a sampling volume of the sampler. The sample material may be obtained through the sample port of the sampler body and into the sampling volume. The receptacle may extend inwards from an outer surface of a cartridge body of the cartridge to a base end of the receptacle. The sample material may be forced out of the sampling volume, into a fluidic channel, and across a sensing surface of a bulk acoustic wave resonator.

In one or more embodiments, forcing the sample material out of the sampling volume may include controlling the flow rate of the sample material out of the sampling volume. In one or more embodiments, inserting the sampler body into the receptacle of the cartridge may include forming a seal between the sample port and a portion of the fluidic channel. In one or more embodiments, the method may further include forcing a buffer fluid from a fluid reservoir and through the fluidic channel to prepare the fluidic channel for the sample material. In one or more embodiments, forcing the buffer fluid from the fluid reservoir and through the fluidic channel further may include passing the buffer fluid across the sensing surface of the bulk acoustic wave resonator to pre-condition the sensing surface. In one or more embodiments, the buffer fluid may be forced from the fluid reservoir at a pressure greater than or equal to a pressure that the sample material is forced out of the sampling volume. In one or more embodiments, the method may further include removing the sampler from the receptacle of the cartridge before obtaining the sample material.

The above summary is not intended to describe each embodiment or every implementation. Rather, a more complete understanding of illustrative embodiments will become apparent and appreciated by reference to the following Detailed Description of Selected Embodiments and Claims in view of the accompanying figures of the drawing.

### BRIEF DESCRIPTION OF THE VIEWS OF THE DRAWING

Exemplary embodiments will be further described with reference to the figures of the drawing, wherein:

FIG. 1 illustrates a schematic perspective view of a system according to an embodiment;

FIG. 2 illustrates a schematic top view of an embodiment of a sampler in accordance with embodiments of the present disclosure;

FIG. 3 illustrates a schematic top view of an embodiment of a cartridge defining a receptacle for receiving the sampler of FIG. 2;

FIG. 4 illustrates the sampler of FIG. 2 received by the receptacle of the cartridge of FIG. 3;

FIG. 5 illustrates a cross-sectional view of an interface between the sampler of FIG. 2 and the cartridge of FIG. 3; and

FIG. 6A-6E illustrate a method of testing a sample material using the sampler of FIG. 2 and the cartridge of FIG. 3.

The figures are rendered primarily for clarity and, as a result, are not necessarily drawn to scale. Moreover, various structure/components may be shown diagrammatically or removed from some or all of the views to better illustrate aspects of the depicted embodiments, or where inclusion of such structure/components is not necessary to an understanding of the various exemplary embodiments described herein. The lack of illustration/description of such structure/components in a particular figure is, however, not to be interpreted as limiting the scope of the various embodiments in any way. Still further, “Figure x” and “FIG. x” may be used interchangeably herein to refer to the figure numbered “x.”

### DETAILED DESCRIPTION OF SELECTED EMBODIMENTS

In the following detailed description, several specific embodiments of devices, systems and methods are disclosed. It is to be understood that other embodiments are contemplated and may be made without departing from the scope or spirit of the present disclosure. Reference is made to the accompanying figures of the drawing which form a part hereof. It is to be understood that other embodiments, which may not be described and/or illustrated herein, are certainly contemplated. The following detailed description, therefore, is not to be taken in a limiting sense.

The present disclosure relates to bulk acoustic wave (BAW) resonators and their use as biosensors. In particular, the present disclosure relates to devices, such as cartridges, containing bulk acoustic wave resonators and configured to receive a sampler including sample material.

As shown schematically in FIG. 1, the cartridge 100 may be used as part of a system 10 with a device or reader 12 constructed to receive the cartridge 100. The reader 12 may provide solutions used in sample handling in the cartridge 100, and may be used to read and optionally interpret the results from the BAW sensor in the cartridge 100. The cartridge 100 of the present disclosure embodies the integrated components necessary to convert a BAW resonator, or array of BAW resonators, into a biosensor. This allows for a collection of the tools integrated into a single cartridge that can be tailored for the detection of one or more analytes, such as proteins, DNA, bacteria, fungi, viruses, and other biological or non-biological materials.

The disclosed cartridges can accommodate a large breadth of testing protocols without requiring the platform to be entirely redesigned. The disclosed cartridges may also provide for the use of the same configuration for different protocols, meaning that only the materials would need to be different to afford different protocols to be undertaken with the device. The cartridges may be manufactured with a selectable or interchangeable sensor platform that allows for even more flexibility. The cartridges or parts of the cartridges may be reusable, recyclable, or disposable. The cartridges may be offered as “dry” cartridges, meaning that no liquid reagents are stored on the device, making the cartridges simpler and more cost-effective to manufacture, and improving storage life of the device. The cartridges are portable and can be used at the sampling location or transported into a laboratory or other secondary site for analysis.

The cartridges of the present disclosure are constructed to receive a liquid sample (e.g., through a sampler received by the cartridge), to at least temporarily store the sample, to provide sample handling and conditioning, and to transfer and meter the sample to a sensor for analysis of one more parameters of the sample. Examples of typical samples include biological samples, such as urine, plasma, serum, blood, saliva, tears, sweat, and the like, and environmental samples, such as air or other gases, water, and aqueous solutions. However, the device can be modified to accommodate various types of fluid samples, and is not particularly limited by sample type.

The cartridges of the present disclosure utilize sensors with bulk acoustic wave (BAW) resonators. According to an embodiment, the cartridge contains a BAW resonator in a fluid flow path. BAW resonators generally include a piezoelectric crystal resonator that can be used to detect changes in material (e.g., changes in the mass of the material) deposited (e.g., bound) on the surface of the resonator or changes in fluid properties (such as viscosity) of a sample. The BAW resonator may have biomolecules, such as antibodies or other proteins such as receptors, or the like, attached to its surface such that when the target analyte passes over the surface, it binds onto the biomolecule. Binding of the analyte the biomolecule attached to the surface of the sensor may increase the mass bound to the sensor, which may alter the wave propagation characteristics (e.g., magnitude, frequency, phase, etc.) of the sensor. The change in propagation characteristics due to analyte binding may be correlated with the amount of bound analyte and, thus, the amount of analyte in the sample. The cartridge may be prepared with various select biomolecules based on the desired target analyte or analytes.

BAW devices typically involve transduction of an acoustic wave using electrodes arranged on opposing top and bottom surfaces of a piezoelectric material. In a BAW device, three wave modes may propagate, namely, one longitudinal mode (embodying longitudinal waves, also called compressional/extensional waves), and two shear modes (embodying shear waves, also called transverse waves), with longitudinal and shear modes respectively identifying vibrations where particle motion is parallel to or perpendicular to the direction of wave propagation. The longitudinal mode is characterized by compression and elongation in the direction of the propagation, whereas the shear modes consist of motion perpendicular to the direction of propagation with no local change of volume. Longitudinal and shear modes propagate at different velocities. In practice, these modes are not necessarily pure modes as the particle vibration, or polarization, is neither purely parallel nor purely perpendicular to the propagation direction. The propagation characteristics of the respective modes depend on the material properties and propagation direction respective to the crystal axis orientations. The ability to create shear displacements is beneficial for operation of acoustic wave devices with fluids (e.g., liquids) because shear waves do not impart significant energy into fluids. BAW devices include bulk acoustic resonators deposited on one or more reflective layers, such as Bragg mirror, and film bulk acoustic resonators having an air-gap.

The BAW sensor described herein may employ any suitable piezoelectric thin film. Certain piezoelectric thin films are capable of exciting both longitudinal and shear mode resonance, such as hexagonal crystal structure piezoelectric materials including (but not limited to) aluminum nitride (AlN) and zinc oxide (ZnO). To excite a wave including a shear mode using a piezoelectric material layer arranged between electrodes, a polarization axis in a piezoelectric thin film is generally non-perpendicular to (e.g., tilted relative to) the film plane. In sensing applications involving liquid media, the shear component of the resonator is preferably used. In such applications, piezoelectric material may be grown with a c-axis orientation distribution that is non-perpendicular relative to a face of an underlying substrate to enable a BAW resonator structure to exhibit a dominant shear response upon application of an alternating current signal across electrodes thereof. Conversely, a piezoelectric material grown with a c-axis orientation that is perpendicular relative to a face of an underlying substrate will exhibit a dominant longitudinal response upon application of an alternating current signal across electrodes thereof.

FIG. 2 illustrates a sampler 200 configured to contain a sample material 202 and configured to be received within the cartridge 100. The sampler 200 may be used for acquiring and delivering a sample material 202 into the cartridge 100 used for, e.g., conditioning and/or analysis of that sample material 202. The sampler 200 may include a sampler body 210 extending between a distal end 212 and a proximal end 214. The sampler body 210 may include an inner surface 216 that defines a sampling volume 218 within the sampler body 210. The sampling volume 218 may be configured to hold or store the sample material 202. The sampling volume 218 may be any suitable size for containing a desired volume of sample material 202. For example, the sampling volume 218 may define a total volume of between about 10 microliters and 100 microliters. Specifically, the sampling volume 218 may define a total volume of about 10 to 20 microliters. In one or more embodiments, it may be described that the total volume of the sampling volume 218 may limit a total volume of the sample material 202 held by the sampling volume 218. In other words, the sample material 202 may be configured to fill up the entirety of the sampling volume 218 and, therefore, the total volume of the sampling volume 218 may control the amount of sample material 202.

The sampler 200 may be configured or adapted to receive the sample material 202 and at least temporarily store and transport the sample material 202 before the sampler 200 is inserted into or received by the cartridge 100, for the sample material 202 to be analyzed. The ability to separate the sampler 200 from the cartridge may provide for an increase in convenience and mobility of the sample material 202 due to the smaller size of the sampler 200 compared to the cartridge 100. For example, the sampler 200 may be easily transported to gather or acquire the sample material 202 (wherever that may be) and then transported or delivered back to the cartridge 100. Additionally, the sampler 200 may minimize the need for additional equipment (e.g., containers, pipettes, etc.) to acquire, contain, and introduce samples. Further, for example, due to the smaller size of the sampler 200 compared to the cartridge 100, multiple samplers 200 may be transported to gather or acquire multiple sample materials 202 and minimize the required storage space due to the reduced size of the sampler 200. In essence, the sampler 200 may be designed to accomplish specific tasks (e.g., acquiring a sample material 202 in the sampling volume 218, being operably coupled to the cartridge 100 and a pump 20 to expel the sample material 202 towards a sensor) such that the size and form factor of the sampler 200 may be reduced for increased mobility.

The sampler 200 may define a sample port 220 proximate the proximal end 214 and a pump connection port 222 proximate the distal end 212. Although, each of the sample port 220 and the pump connection port 222 may be positioned at any suitable location on the sampler body 210. Each of the sample port 220 and the pump connection port 222 may be in fluid communication with the sampling volume 218. The sample port 220 may be an entry point for the sample material 202 to enter the sampling volume 218, as well as an exit point for the sample material 202 to be dispersed out of the sampling volume 218. Further, the sample port 220 may be configured to be operably connected to a fluidic channel 140 of the cartridge 100 (e.g., when the sampler 200 is received by the cartridge 100 as shown in FIG. 4). In other words, the sample port 220 may be in fluid communication with the fluidic channel 140 of the cartridge 100 such that the sample material 202 may be forced out of the sampling volume 218 and into the fluidic channel 140 (e.g., so that the sample material 202 may be analyzed by a sensor located in the cartridge 100).

In one or more embodiments, the inner surface 216 of the sampler body 210 may include a hydrophilic material configured to draw the sample material 202 into the sampling volume 218 through the sample port 220. In other words, the inner surface 216 defining the sampling volume 218 may include a hydrophilic material to attract (e.g., through a wicking effect or capillary action) the sample material 202 such that the sample material 202 enters the sampling volume 218 through the sample port 220. Additionally, the use of a hydrophilic material may help with preventing excess air in the sampling volume 218 with the sample material 202. Further, in one or more embodiments, the sampler 200 may also include a puncture portion located proximate the sample port 220. For example, in some embodiments, the puncture portion may be removably couplable to the proximal end 214 of the sampler 200 proximate the sample port 220 (e.g., coupled to the sampler 200 when drawing the sample material 202 and removed from the sampler 200 before inserting the sampler 200 into the cartridge 100). The puncture portion may be configured to puncture a barrier 5 (e.g., skin as shown in FIG. 6B) to access the sample material 202 and allow the sample material 202 to be collected into the sampling volume 218. The puncture portion may include any suitable components for puncturing the barrier to access the sample material 202. For example, the puncture portion may include subcutaneous hollow microneedles of either metallic or polymer materials, hypodermic needles, or the like.

The sample material 202 may be retained within the sampling volume 218 of the sampler 200 due to, for example, surface tension forces at the liquid/solid/air interface of the sample port 220. In one or more embodiments, a cap may cover the sample port 220 at the proximal end 214 of the sampler 200 to prevent sample material 202 from exiting the sampler 200 before desired and to help minimize evaporation and concentration at the sample port 220. The cap may then be removed from the sampler 200 prior to inserting the sampler 200 into the cartridge 100. In some embodiments, the sampler may be configured to be immediately placed into the cartridge 100 after the sampler material 202 is acquired by the sampler 200 to help minimize evaporation and concentration at the sample port 220.

The pump connection port 222 of the sampler 200 may be configured to be operably connected to a pump 20 (e.g., as shown in FIG. 4) to push or force the sample material 202 out of the sampling volume 218. Accordingly, the pump connection port 222 (e.g., located opposite the sample port 220) may provide access to the sampling volume 218 such that the pump 20 operably coupled to the pump connection port 222 may apply a pneumatic or hydraulic force (e.g., through the pump connection port 222) to the sample material 202, thereby forcing the sample material 202 out of the sample port 220. Further, the force (e.g., pneumatic or hydraulic) applied to the pump connection port 222 by the pump 20 may force the sample material 202 from the sampling volume 218 at a controlled flow rate. In some embodiments, it may be desirable to control the flow rate of the sample material 202 exiting the sampler 200 and traveling over the resonator sensor based on the response of the sensor. For example, if the sensor response is indicative of a low concentration of analyte in the sample, it may be beneficial to decrease the rate of flow to increase the opportunity for the analyte to bind to a biomolecule on the surface of a resonator.

The sampler 200 may further include a barrier or wall (e.g., similar to a piston) positioned between the sample material 202 and the pump connection port 222. The barrier or wall may be provided to separate the sample material 202 located in the sampling volume 218 from any gas or fluid that may be inserted through the pump connection port 222 (e.g., by the pump 20) to force the sample material 202 out of the sampling volume 218. The barrier or wall may provide a physical barrier between the sample material 202 and the gas or fluid inserted into the sampling volume 218 to prevent undesired mixing of the that gas or fluid and the sample material 202. The barrier or wall may include a polymer material with hydrophobic properties to help prevent liquid from leaking around the wetted perimeter. Furthermore, the barrier or wall may be free floating such that the barrier or wall moves along with the sample material 202 as the sampling volume 218 is filled or depleted.

Furthermore, the pump connection port 222 may include a vent 224 positioned between the sampling volume 218 and the exterior environment. The vent 224 may provide a way to relieve pressure from within the sampling volume 218 when the sample material 202 is drawn into the sampling volume 218 from the sample port 220 (e.g., similar to a pressure release valve on a container). The vent 224 may include a hydrophobic material such that the sample material may be prevented from passing through the vent 224 and, e.g., out of the sampling volume 218.

A cartridge 100 that may receive the sampler 200 is illustrated in FIG. 3. The cartridge 100 may include a cartridge body 110 defining a receptacle 114 extending inwards from an outer surface 112 of the cartridge body 110 to a base end 116 of the receptacle 114. The receptacle 114 may be configured to receive the sampler 200 (e.g., the proximal end 214) such that the sampler 200 may be removably couplable to the cartridge 100. In other words, the sampler 200 may be configured to be coupled within the cartridge 100, removed from the cartridge 100, and coupled again to the cartridge 100 without damaging the integrity of either the sampler 200 or the cartridge 100. As shown in FIG. 4, the sampler 200 may be sized relative to the cartridge 100 such that the distal end 212 of the sampler 200 is flush with the outer surface 112 of the cartridge body 110.

Additionally, the sampler 200 may be configured to lock or secure into position within the receptacle 114 of the cartridge body 110. In other words, the sampler 200 may be configured such that the sampler cannot fall or slip out of the receptacle 114 after the sampler 200 has been received by the receptacle 114. In some embodiments, the sampler 200 and the receptacle 114 may be sized such that there is an interference fit between the sampler 200 and the receptacle 114 (e.g., friction may help maintain the sampler 200 attached within the receptacle 114). In other embodiments, the sampler 200 and the receptacle 114 may include a locking apparatus to maintain a robust connection between the sampler 200 and the receptacle 114 when the sampler 200 is received by the receptacle 114. For example, sampler 200 and the receptacle 114 may interact through a snap hook mechanism, a luer lock, threads, a bayonet mount, a clip, or any other suitable fastener.

The cartridge 100 may include a bulk acoustic wave resonator sensor 120 as described herein. The bulk acoustic wave resonator sensor 120 may include a bulk acoustic wave resonator 122 having a sensing surface (e.g., an analyte-binding surface). The resonator sensor 120 depicted in FIG. 3 includes a plurality of bulk acoustic wave resonators 122 located in a fluid flow path 140 through which an acquired sample material may flow. The sensor 120 may have one or more electronic components to drive the resonators 122 into oscillating motion and measure a change in an oscillation characteristic of the resonator 122 as the sample material is passed over the sensing surface of the resonators 122. In addition or alternatively, one or more electronic components for driving and sensing a response from the resonators 122 may be external to the cartridge, such as on a reader 12 depicted in FIG. 1. The reader or other suitable external device may be electrically coupled to the sensor 120 via electrical interconnect 129.

The resonators 122 of the sensor 120 may be used for a variety of different suitable purposes. For example, the at least one sensor 120 may be used in a control group or to provide redundancy.

The cartridge 100 includes a fluidic channel 140 (e.g., microfluidics) configured to carry fluid at least between the receptacle 114 and the sensor 120. The fluidic channel 140 may be any suitable passageway through which a fluid (e.g., the sample material 202) may travel. The fluidic channel 140 may be made from or coated with hydrophobic or hydrophilic materials to optimize or control the ability for fluids to flow through the fluidic channel 140.

The fluidic channel 140 may include multiple fluidic channel portions extending throughout the cartridge body 110 and aligned to transport fluid to and from the sensor 120. For example, the fluidic channel 140 may include a first fluidic channel portion 141 extending between a reservoir connection port 134 and the receptacle 114. The first fluidic channel portion 141 may be positioned upstream of the receptacle 114 such that fluid is configured to travel through the first fluidic channel portion and towards the receptacle 114.

The fluidic channel 140 may also include a second fluidic channel portion 142 extending from the first fluidic channel portion 141 and proximate the base end 116 of the receptacle 114. The second fluidic channel portion 142 may be in fluid communication with the sample port 220 of the sampler 200 when the sampler 200 is received by the receptacle 114 (e.g., as shown in FIG. 4). For example, the sampler 200 and the receptacle 114 may form a seal when the sampler 200 is received by the receptacle 114 to form a fluid-tight interface between the sample port 220 and the second fluidic channel portion 142. In some embodiments, at least a portion of the second fluidic channel portion 142 may be formed by the sampler 200 when the sampler 200 is received by the receptacle 114. In other words, the second fluidic channel portion 142 may extend coextensive along the base end 116 of the receptacle 114 to form a path for fluid to travel.

FIG. 5 illustrates one embodiment of the interface between the sampler 200 and the cartridge 100. For example, the sampler 200 may be inserted into the cartridge 100 and sized such that the sampler 200 may form an interference fit with the cartridge 100. For example, the attachment portion 115 of the cartridge 100 may contact the sampler 200 to create friction between the sampler 200 and the attachment portion 115 such that the sampler 200 may be secured in the cartridge 100. Further, the proximal end 214 of the sampler 200 may form a portion of the second fluidic channel portion 142 such that fluid flowing from the first fluidic channel portion 141 may contact the sampler 200 and proceed towards the third fluidic channel portion 143. Furthermore, the proximal end 214 of the sampler 200 may extend into the cartridge 100 such that the sample material 202 exiting the sampling volume 218 may only flow towards the third fluidic channel portion 143. As a result, the sample material 202 may be prevented from flowing backwards towards the first fluidic channel portion 141 and, similarly, fluid flowing from the first fluidic channel portion 141 may flow through the second fluidic channel portion 142 to the third fluidic channel portion 143 and be prevented from flowing backwards into the sampling volume 218.

In some embodiments, the sampler 200 or receptacle 114 of the cartridge 100 may include an over molded portion or gasket to help form a seal between the sampler 200 and the cartridge 100 when the sampler 200 is inserted therein. Furthermore, in some embodiments, the sample port 220 of the sampler 200 may include a hydrophobic barrier or valve to prevent premature flow of sample material 202 out of the sampler 200 (e.g., when the sampler 200 is received by the receptacle 114) and which may be overcome by pressure applied to the pump connection port 222 such that the force applied pushes the sample material 202 through the hydrophobic barrier or valve. Additionally, in one or more embodiments, during the introduction and flow of the buffer material 132 in the cartridge 100, the buffer material 132 may be prevented from entering the sample port 220 by the application of pressure to the pump connection port 222 (e.g., to apply pressure to sample material 202 in the sampling volume 218). On the other hand, to flow only the sample material 202 towards the sensor 120, a pressure may be applied to the buffer material 132 to maintain no flow within the first fluidic channel portion 141 of the buffer material 132.

The fluidic channel 140 may also include a third fluidic channel portion 143 extending between the second fluidic channel portion 142 and the (resonator) sensor 120. The third fluidic channel portion 143 may be positioned downstream of the receptacle 114 such that fluid may travel from the second fluidic channel portion 142 and towards the sensor 120. For example, sample material 202 from the sampler 200 may be forced out of the sampling volume 218 and towards the sensor 120. Further, any fluid (e.g., buffer material) passing through the first fluidic channel portion 141 may also travel through the third fluidic channel portion 143 to the sensor 120.

Furthermore, the third fluidic channel portion 143 may include a mixing region 149 for the sample material 202 to mix with a mixing fluid (e.g., buffer material 132). The mixing may occur downstream of the sampler 200 when inserted into the cartridge 100 and upstream of the sensor 120. This location of the mixing region 149 may allow the sample material 202 to be adequately mixed before reaching the one or more resonators 122 of the sensor 120. The sample material 202 may be mixed with the mixing fluid to, e.g., dilute the sample material 202 or mix the sample material 202 with buffering agents or reagents. To mix the sample material 202 with the buffer material 132, an appropriate ratio of pressures may be applied to both the sample material 202 (e.g., through the pump connection port 222) and the buffer material 132 (e.g., through the reservoir connection port 134).

The fluidic channel 140 may further include a fourth fluidic channel portion 144 extending from the third fluidic channel portion 143 and across the sensing surface of the one or more resonators 122 of the sensor 120. The fourth fluidic channel portion 144 may be positioned such that the buffer material 132 may pass over the one or more resonators 122 and/or the sample material 202 may pass over/be deposited on the one or more resonators 122.

In one or more embodiments, the cartridge 100 may include the reservoir connection port 134 to introduce a buffer material 132 into the fluidic channel 140 (e.g., at the first fluidic channel portion 141). For example, the buffer material 132 may pass through the fluidic channel 140 to prime the fluidic channel 140 (e.g., to prevent any air bubbles from presenting in the fluidic channel 140). Further, the buffer material 132 may pass through the fluidic channel 140 to clean or wet the sensing surface of the resonators 122 as the buffer material 132 passes over the resonators 122.

The cartridge 100 optionally includes a fluid reservoir 130 configured to hold the buffer material 132. For example, as shown in FIG. 3, the fluid reservoir 130 is located along the length of the first fluidic channel portion 141 between the reservoir connection port 134 and the receptacle 114. Therefore, instead of the buffer material 132 being introduced to the cartridge 100 from a source exterior to the cartridge 100, the fluid reservoir 130 stores a volume of buffer material 132 within the cartridge 100 (e.g., from the device or reader 12, through a port, etc.). The fluid reservoir 130 may define a total volume of any suitable size. Specifically, the fluid reservoir 130 may define a total volume of about up to 1 mL. The first fluid channel portion 141 may be in fluid communication with the fluid reservoir 130 such that the first fluid channel portion 141 extends into and extends out of the fluid reservoir 130. Further, the reservoir connection port 134 may be in fluid communication with the fluid reservoir 130, e.g., via the first fluid channel portion 141 (e.g., to apply a force to the reservoir connection port 134 to force the buffer material 132 out of the fluid reservoir 130).

In one or more embodiments, the cartridge 100 may include a sealed fluid portion 135 (e.g., a reagent blister) configured to store buffer material 132 in a sealed compartment prior to use of the cartridge 100. The sealed fluid portion 135 is specifically shown in FIG. 3, however, may be implemented with any cartridge 100 described and depicted herein. By storing the buffer material 132 within the sealed fluid portion 135, the buffer material 132 may not prematurely flow through the fluidic channel 140 before it is intended. Once the sampler 200 is inserted into the receptacle 114 of the cartridge 100 (and, e.g., the cartridge 100 is positioned within the device or reader 12), the sealed fluid portion 135 may be actuated (e.g., broken) such that the buffer material 132 exits the sealed fluid portion 135 and into the fluid reserveroir.130. From there, the buffer material 132 may move into the fluidic channel 140 as desired. The sealed fluid portion 135 may be actuated by insertion of the cartridge 100 into the device or reader 12, by an internal component of the device or reader 12, or in any other suitable way.

In one or more embodiments, the cartridge 100 may also include a waste container 160 downstream of the sensor 120 (e.g., at an end of the fluidic channel 140). The waste container 160 may be used to collect waste material (e.g., sample material 202, buffer material 134, any other fluids used to process the sample, etc.) that has already passed over the sensor 120. The waste container 160 may be any suitable size to hold the various fluids to dispose of within the cartridge 100 (e.g., at least larger than the fluid reservoir 130 and the sampling volume 218 combined). For example, the waste container 160 may define a total volume of between about 100 microliters and 2 milliliters. Further, the waste container 160 may include a waste vent 164 that allows air or gases, but not fluids, to escape the waste container 160 in order to, e.g., equalize pressure within the waste container 160. The waste vent 164 may include a hydrophobic membrane to prevent fluid from passing through the waste vent 164.

Additionally, the fluidic channel 140 may further include a fifth fluidic channel portion 145 extending between the fourth fluidic channel portion 144 and the waste container 160. The fifth fluidic channel portion 145 may be in fluid communication with an inlet 162 of the waste container 160 such that fluid traveling through the fifth fluidic channel portion 145 may pass into the waste container 160.

As shown in FIG. 4, the system 10 may also include a pump 20 (e.g., a displacement pump). The pump 20 may be operably coupled to the pump connection port 222 to force the sample material 202 out of the sampling volume 218. Further, the pump 20 may also be operably coupled to the reservoir connection port 134 to force buffer material 132 through the fluidic channel 140 (e.g., through pneumatics or hydraulics). The system 10 may include a valve 24 (e.g., illustrated in broken lines in FIG. 4 because the valve 24 may or may not be included in the system) operably coupled between the pump 20 and each of the pump connection port 222 and the reservoir connection port 134. The valve 24 (e.g., a metering valve) may be configured to selectively control pressure from the pump 20 to each of the sampling volume 218 via the pump connection port 222 and the reservoir connection port 134. For example, the valve 24 may allow for the selection a different pressure to be applied to each of the sampling volume 218 of the sampler 200 and the reservoir connection port 134. It may be desirable to apply a different pressure to each of the sampling volume 218 of the sampler 200 and the reservoir connection port 134 as described further herein.

In some embodiments, the system 10 may include a second pump 22 (e.g., illustrated in broken lines in FIG. 4 because the second pump 22 may or may not be included in the system) configured to be operably coupled to the reservoir connection port 134 to force buffer material 132 through the fluidic channel 140 (e.g., out of the fluid reservoir 130) by, e.g., pneumatics or hydraulics. Furthermore, regardless of whether or not the valve 24 and the pump 20 are implemented or the second pump 22 is implemented, pressure applied (e.g., by the pump 20) to force the sample material 202 out of the sampling volume 218 of the sampler 200 may be less than or equal to a pressure applied (e.g., by the second pump 22 or by the pump 20 through the valve 24) to force the buffer material 132 through the fluidic channel 140. In other words, the buffer material 132 may be driven through the fluidic channel 140 at a greater than or equal pressure to the sample material 202 being driven out of the sampler 200. By controlling the pressure differentials in this way, the system 10 may prevent the sample material 202 from being forced through the fluidic channel 140 in the wrong direction (e.g., upstream).

Furthermore, the pump 20 may be configured to apply a force (e.g., pneumatic or hydraulic) through the pump connection port 222 of the sampler 200 to force the sample material 202 out of the sampling volume 218 at a controlled flow rate. As described herein, it may be desirable to control the flow rate of the sample material 202 exiting the sampler 200 and traveling over the sensor 120 based on the response of the sensor 120.

The system 10 may include a controller 26 to control the pumps 20, 22 and valve 24 that may be present in the system. Furthermore, each of the pumps 20, 22, controller 26, and valve 24 may be contained within the device or reader 12. The methods and/or logic described in this disclosure, including those attributed to the system 10, or various constituent components (e.g., the controller 26), may be implemented, at least in part, in hardware, software, firmware, or any combination thereof. For example, various aspects of the techniques may be implemented within one or more processors, including one or more microprocessors, microcontrollers, DSPs, ASICs, FPGAs, or any other equivalent integrated or discrete logic circuitry, as well as any combinations of such components, or other devices. The term “processor” or “processing circuitry” may generally refer to any of the foregoing logic circuitry, alone or in combination with other logic circuitry, or any other equivalent circuitry. Such hardware, software, and/or firmware may be implemented within the same system or within separate systems to support the various operations and functions described in this disclosure. In addition, any of the described components may be implemented together or separately as discrete but interoperable logic devices.

When implemented in software, the functionality ascribed to the systems, devices and methods described in this disclosure may be embodied as instructions and/or logic on a computer-readable medium such as RAM, ROM, NVRAM, EEPROM, FLASH memory, magnetic data storage media, optical data storage media, or the like. The instructions and/or logic may be executed by one or more processors to support one or more aspects of the functionality described in this disclosure.

When the cartridge 100 is operably coupled to external equipment of the system 10 (e.g., within the device or reader 12), the sensor 120 may be electrically coupled to the controller 26 via electrical interconnect 129. The sensor 120, external device, and controller 26 include one or more electronic components to drive the resonators 122 into oscillating motion and measure a change in an oscillation characteristic of the resonator as the sample material is passed over the sensing surface of the resonators 122.

An example of a method of testing a sample material 202 is illustrated in FIGS. 6A-6E. However, the method may first include providing a cartridge 100 with a sampler 200 (e.g., an unused sampler 200 containing no sample material 202) inserted therein, as shown in FIG. 4. Thereafter, the method may include removing the sampler 200 from the receptacle 114 of the cartridge 100 before obtaining the sample material 202, as shown in FIG. 6A. In order to remove the sampler 200 from the receptacle 114, in some embodiments, the sampler 200 may be unlocked or disengaged such that the sampler 200 may move freely relative to the cartridge body 110. In some embodiments, the sampler 200 may be initially provided separate from the cartridge 100.

Next, the method may include obtaining the sample material 202 in the sampler 200 through the sample port 220 of the sampler body 210 and into the sampling volume 218, as shown in FIG. 6B. In one or more embodiments, obtaining the sample material 202 in the sampling volume 218 may include puncturing a barrier 5 (e.g., skin) to access the sample material 202. Further, obtaining the sample material 202 may include drawing (e.g., wicking) the sample material 202 through a hydrophilic material of the sampling volume 218 as described herein. In one or more embodiments, a cap may be positioned over the sample port 220 of the sampler 200 to prevent sample material 202 from exiting the sampling volume 218 while the sampler 200 is in transport.

The method may also include inserting the sampler body 210 into a receptacle 114 of a cartridge 100, as shown in FIG. 6C. As described herein, the receptacle 114 may extend inwards from an outer surface 112 of a cartridge body 110 to a base end 116 of the receptacle 114. The sampler body 210 may be inserted into the receptacle 114 such that the sampler 200 may be “locked” or secured into place within the receptacle 114 (e.g., to prevent inadvertent removal of the sampler 200). Further, the sampler body 210 may be inserted into the receptacle 114 such that a seal forms between the sample port 220 and a portion of the fluidic channel 140. In some embodiments, fully inserting the sampler body 210 into the receptacle 114 may form a portion of the fluidic channel 140 proximate the base end 116 of the receptacle 114. In one or more embodiments, the sampler body 210 may be inserted into the receptacle 114 to a point in which a portion of the sampler body 210 may be flush with the outer surface 112 of the cartridge body 110.

As shown in FIG. 6D, the method may also include forcing a buffer fluid 134 (e.g., from a fluid reservoir 130 within the cartridge 100) through the fluidic channel 140 to prepare or prime the fluidic channel 140 for the sample material 202. In some embodiments, forcing the buffer fluid 134 from the fluid reservoir 130 and through the fluidic channel 140 may include passing the buffer fluid 134 across the resonators 122 of the sensor 120 to pre-condition the resonators 122. Further, in some embodiments, the buffer fluid 134 may be forced through the fluidic channel 140 at a pressure greater than or equal to a pressure that the sample material 202 is forced out of the sampling volume 218. Forcing the buffer material 134 at a greater than or equal pressure to the sample material 202 may help to prevent the sample material 202 from flowing upstream (e.g., away from the resonator sensor 120).

Further, the method may include forcing the sample material 202 out of the sampling volume 218, into the fluidic channel 140, and across the one or more resonators 122 of the sensor 120, as shown in FIGS. 6D-6E. For example, in some embodiments, after the sampler 200 is inserted into the cartridge 100, the cartridge 100 may be inserted into the device or reader 12 (e.g., as shown in FIG. 1 and the broken outline of the device or reader 12 is shown in FIG. 6E). The pump connection port 220 may then be operably connected to the pump 20 due to this insertion of the cartridge 100 (including the sampler 200) into the device or reader 12. Further, the resonator sensor 120 may also be operably connected to the controller 26 when the cartridge is inserted into the device or reader 12. After the cartridge 100 is inserted into the device or reader 12 and operably coupled to the components therein, the sample material 202 may be forced out of the sampling volume 218 such that the sample material 202 may be analyzed.

In one or more embodiments, forcing the sample material 202 out of the sampling volume 218 may include applying a force through the pump connection port 222 of the sampler body 210 that is in fluid communication with the sampling volume 218. Further, forcing the sample material 202 out of the sampling volume 218 may include controlling the flow rate of the sample material 202 out of the sampling volume 218. Controlling the flow rate of the sample material 202 out of the sampling volume 218 and over the sensor 120 may help to provide consistent results. Further yet, the force may be applied by the pump 20 (e.g., which may be operably coupled to the pump connection port 220 due to inserting the cartridge 100 into the device or reader 12).

In one or more embodiments, the method may further include mixing the sample material 202 with a mixing fluid before forcing the sample material 202 across the resonator 122 of the sensor 120. In one or more embodiments, the method may also include forcing the sample material 202 across a sensing surface of one or more resonators 122.

Illustrative embodiments are described and reference has been made to possible variations of the same. These and other variations, combinations, and modifications will be apparent to those skilled in the art, and it should be understood that the claims are not limited to the illustrative embodiments set forth herein.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open-ended sense, and generally mean “including, but not limited to.” It will be understood that “consisting essentially of” “consisting of,” and the like are subsumed in “comprising” and the like. As used herein, “consisting essentially of” as it relates to a composition, product, method or the like, means that the components of the composition, product, method or the like are limited to the enumerated components and any other components that do not materially affect the basic and novel characteristic(s) of the composition, product, method or the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

1. A sampler comprising:
a sampler body extending between a distal end and a proximal end, wherein the sampler body comprises an inner surface defining a sampling volume within the sampler body that is configured to hold a sample material, wherein the sampler body defines a sample port proximate the proximal end and a pump connection port proximate the distal end, wherein each of the sample port and the pump connection port are in communication with the sampling volume,
wherein the sample port is configured to be operably connected to a fluidic channel of a cartridge,
wherein the pump connection port is configured to be operably connected to a pump.

2. The sampler of claim 1, wherein the inner surface of the sampler body comprises a hydrophilic material configured to draw the sample material into the sampling volume through the sample port.

3. The sampler of claim 1, wherein the pump connection port comprises a vent including a hydrophobic material such that the sample material is prevented from passing through the vent.

4. The sampler of claim 1, wherein the sampler is configured to force the sample material from the sampling volume through the sample port in response to pneumatic forces applied through the pump connection port by the pump.

5. The sampler of claim 1, wherein the pneumatic forces applied to the pump connection port by the pump force the sample material from the sampling volume at a controlled flow rate.

6. The sampler of claim 1, wherein a total volume of the sampling volume limits a total volume of the sample material held by the sampling volume, wherein the total volume is between about 10 microliters to 100 microliters.

7. The sampler of claim 1, further comprising a puncture portion located proximate the sample port and configured to puncture a barrier.

8. A system comprising:
the sampler of claim 1; and
a cartridge comprising:
a cartridge body defining a receptacle extending inwards from an outer surface of the cartridge body to a base end of the receptacle, wherein the receptacle is configured to receive the sampler such that the sampler is removably couplable to the cartridge,
a sensor comprising a bulk acoustic wave resonator having a sensing surface, and
a fluidic channel configured to carry fluid at least between the receptacle and the sensor, wherein the fluidic channel comprises:
a first fluidic channel portion extending between a reservoir connection port and the receptacle,
a second fluidic channel portion extending from the first fluidic channel portion and proximate the base end of the receptacle, wherein the second fluidic channel portion is in fluid communication with the sample port of the sampler when the sampler is received by the receptacle,
a third fluidic channel portion extending between the second fluidic channel portion and the sensor, and
a fourth fluidic channel portion extending from the third fluidic channel portion and across the sensing surface of the resonator.

9. The system of claim 8, wherein at least a portion of the second fluidic channel portion is formed by the sampler when the sampler is received by the receptacle.

10. The system of claim 8, wherein the cartridge further comprises a fluid reservoir configured to hold a buffer material, wherein the first fluidic channel portion is in fluid communication with the fluid reservoir, wherein the fluidic channel is configured to carry the buffer material from the fluid reservoir to at least the sensor, wherein the cartridge further comprises the reservoir connection port in fluid communication with the fluid reservoir.

11. The system of claim 10, wherein the pump is operably coupled to the pump connection port to force the sample material out of the sampling volume and operably coupled to the reservoir connection port to force the buffer material through the fluidic channel, wherein the system further comprises a valve operably coupled between the pump and each of the pump connection port and the reservoir connection port, wherein the valve is configured to selectively control pressure from the pump to each of the sampling volume and the fluid reservoir.

12. The system of claim 10, wherein the pump is configured to be operably coupled to the pump connection port to force the sample material out of the sampling volume, wherein the system further comprises a second pump configured to be operably coupled to the reservoir connection port to force the buffer material through the fluidic channel, wherein a pressure applied by the pump to the sampling volume to force the sample material out of the sampling volume is less than or equal to a pressure applied by the second pump to the fluid reservoir to force the buffer material through the fluidic channel.

13. The system of claim 8, wherein the pump is configured to apply a force through the pump connection port of the sampler to force the sample material out of the sampling volume at a controlled flow rate.

14. The system of claim 8, wherein the sampler and the receptacle form a seal when the sampler is received by the receptacle to form a fluid-tight interface between the sample port and the second fluidic channel portion.

15. The system of claim 8, wherein the cartridge further comprises a waste container and the fluidic channel further comprises a fifth fluidic channel portion, wherein the fifth fluidic channel portion extends between the fourth fluidic channel portion and the waste container, wherein the fifth fluidic channel portion is in fluid communication with an inlet of the waste container.

16. The system of claim 15, wherein the waste container comprises a waste vent.

17. The system of claim 8, wherein the sampler is configured to lock into position within the receptacle of the cartridge body.

18. The system of claim 8, wherein the cartridge comprises at least one additional bulk acoustic wave resonator, wherein the fluidic channel is configured to carry the sample material across the at least one additional resonator.

19. A method of testing a sample material, the method comprising:
obtaining the sample material in the sampler of claim 1 through the sample port of the sampler body and into the sampling volume;
inserting the sampler body into a receptacle of a cartridge, wherein the receptacle extends inwards from an outer surface of a cartridge body to a base end of the receptacle; and
forcing the sample material out of the sampling volume, into a fluidic channel, and across a sensing surface of a bulk acoustic wave resonator.

20. The method of claim 19, wherein forcing the sample material out of the sampling volume comprises controlling the flow rate of the sample material out of the sampling volume.

21-32. (canceled)
